(19) United States
(12) Patent Application Publication
(10) Pub. No.: US 2008/0200969 A1
(43) Pub. Date: Aug. 21, 2008

(54) TEMPERATURE SENSING APPARATUS AND METHODS FOR TREATMENT DEVICES USED TO DELIVER HIGH FREQUENCY ENERGY TO TISSUE

(75) Inventor: Bryan Weber, Livermore, CA (US)

Correspondence Address: WOOD, HERRON & EVANS, LLP, 2700 CAREW TOWER, 441 VINE STREET, CINCINNATI, OH, 45202, US

(73) Assignee: THERMAGE, INC., Hayward, CA (US)

(21) Appl. No.: 12/032,025

(22) Filed: Feb. 15, 2008

Related U.S. Application Data
(60) Provisional application No. 60/890,295, filed on Feb. 16, 2007.

(51) Int. Cl. A61B 18/18

(52) U.S. Cl. 607/102

(57) ABSTRACT

Apparatus and methods for delivering high frequency energy to tissue with improved temperature sensing. The treatment apparatus may be a delivery device positionable adjacent to the tissue. The delivery device may further include an electrode adapted to deliver high frequency energy to the tissue and at least one thermal sensor. In one embodiment, the thermal sensor may include a thermocouple junction of dissimilar metals formed by either thin film or thick film techniques. Alternatively, the thermal sensor may include a body composed of a resistive material having a resistance that varies with temperature to an extent sufficient to measure the skin temperature. A region of the delivery device near the thermal sensor may be heated, before skin contact is established during treatment, for purposes of detecting contact by the occurrence of heat loss from the delivery device region.

### FIELD OF THE INVENTION

This application claims the benefit of U.S. Provisional Application No. 60/890,295, filed Feb. 16, 2007, which is hereby incorporated by reference herein in its entirety.

### FIELD OF THE INVENTION

The invention generally relates to apparatus and methods for treating tissue with high frequency energy and, more particularly, relates to apparatus and methods for delivering high frequency energy and thermal sensing associated with such apparatus and methods.

### BACKGROUND OF THE INVENTION

Devices that can treat tissue non-invasively are extensively used to treat numerous diverse skin conditions. Among other uses, non-invasive energy delivery devices may be used to tighten loose skin to make a patient appear younger, remove wrinkles and fine lines, contour the skin, remove skin spots or hair, or kill bacteria. Such non-invasive energy delivery devices emit electromagnetic energy in different regions of the electromagnetic spectrum for tissue treatment. Specifically, non-invasive energy delivery devices may treat tissue with ultraviolet, visible, and infrared light, both incoherent and coherent; microwave and radio-frequency (RF) energy; and sonic and mechanical energy.

High frequency treatment devices, such as RF-based devices, may be used to treat skin tissue non-ablatively and non-invasively by passing high frequency energy through a surface of the skin, while actively cooling the skin to prevent damage to a skin epidermis layer. The high frequency energy heats tissue beneath the epidermis to a temperature sufficient to denature collagen, which causes the collagen to contract and shrink and, thereby, tighten the tissue. Treatment with high frequency energy also causes a mild inflammation. The inflammatory response of the tissue causes new collagen to be generated over time (between three days and six months following treatment), which results in further tissue contraction and tissue tightening.

Modern high frequency treatment devices employ multiple discrete temperature sensors whose sensor packages are mounted on and attached to an electrode assembly for ostensively monitoring the temperature of the treatment tip of the high frequency device. Common temperature sensors used in this application are a set of thermistors whose thermistor packages are surface mounted to a non-patient side of the high frequency electrode of the treatment tip. Thermistors are temperature sensors that have resistances that vary with the temperature level. Hence, a temperature change of the thermistor is reflected by a change in either the current through or voltage drop across the thermistor. Such discrete sensor packages are typically relatively large, for example on the order of 500 microns (20 mils).

Among other purposes, the output of the temperature sensors is used for closed-loop control of coolant application and/or detecting aberrant skin temperatures as a safety precaution. In the latter regard, the delivery of high frequency energy to the electrode may be aborted or titrated. The output from the temperature sensors distributed across the treatment tip may also be used to determine if the treatment tip has a flush or canted contact with the skin. For example, changes in the output from temperature sensors at the four corners of a rectangular treatment tip may be used to determine if the four corners are contacting the skin surface during treatment or before the electrode is energized to initiate treatment.

Conventional thermistors measure the temperature of the thermistor and thermistor package. Consequently, the temperature readings from the thermistors may not be representative of, or reflect, the actual temperature of adjacent structures, such as the treatment tip or the patient's skin. The temperature readings of the thermistor are affected by many factors, including but not limited to thermal mass or inertia of the thermistor, the temperature of conductive traces coupled with the thermistor to provide electrical signal paths with a controller, the temperature of the skin near the thermistor, and the temperature of the nearby metal RF electrode. These influences may slow the thermal response of the thermistor and degrade the accuracy of the estimate of the skin temperature.

The non-patient side of the electrode in the electrode assembly in the treatment tip, on which the thermistors are conventionally situated, may be sprayed with a coolant or cryogen spray under feedback control of the thermistors for cooling the skin contacting the electrode assembly. The controller triggers the coolant spray based upon an evaluation of the temperature readings from the thermistors. The temperature readings from the thermistors are dependent upon, among other factors, the spray pattern of the cryogen, any pooling of cryogen near or over the thermistor, and the evaporation rate of any cryogen wetting the thermistor.

The limited isolation of the thermistors from the cryogen introduces errors into determinations of the skin temperature from the temperature readings of the treatment tip temperature. Hence, overheating of the patient's skin may not be detected in a timely manner during the delivery of high frequency energy. The undesirable result is that skin damage may occur before measures are taken to indicate the occurrence of overheating to the clinician or to otherwise remedy the overheating. Moreover, inaccuracies in the detected changes in skin temperature may result in poor control over the timing of individual pulses of cryogen spray directed toward the electrode. Large differences between the thermal mass of the thermistor and the thermal mass of the thin electrode may precipitate a large temperature difference between the thermistor, on one hand, and the electrode assembly and its electrode, on the other hand. For example, a spray of cryogen may reduce the temperature of the electrode by 50° C. and the temperature of the thermistor by only 5° C. Because the controller operates under the assumption that the temperature measured by the thermistor is nominally representative of the electrode and skin temperatures, the electrode may be sprayed prematurely with cryogen because this fundamental assumption is incorrect.

One potential approach for improving the operation of the thermistors is to place the thermistors on the patient side of the electrode assembly such that the thermistors actually contact the skin surface. However, the package for a surface-mounted thermistor would present an irregularity or bump in the otherwise substantially planar patient-contacting surface. A typical package thickness for a surface mount thermistor is on the order of about 20 mils (approximately 0.5 mm or 500 μm). Although the thermistor may be isolated from the artifacts caused by direct contact with the cryogen, the surface irregularity would be evident to the patient. Hence, this acts to limit thermistor placement within the treatment tip. Consequently, the thermistors are conventionally placed on the non-patient facing surface of the electrode in conventional treatment tips.

With regard to contact measurements, the controller for the treatment device may incorporate a lifted algorithm that relies on the temperature readings from the thermistors to determine if one or more edges are lifted out of contact with the patient's skin when high frequency power is applied to the electrode. As a result, the application of power is discontinued to the electrode. When a thermistor is lifted above the skin, the measured temperature rapidly changes to reflect the loss of skin contact. If the thermistor is at the temperature of the patient's skin, the change in thermistor temperature because of an out-of-contact condition may be small. This limits the effectiveness of the software algorithm in responding to a condition in which one or more edges of the electrode have a non-contacting relationship with the skin when the electrode is energized. Heating or cooling of the skin temperature during treatment may also contribute to limiting the response effectiveness of the software lifted algorithm. An initial temperature difference may be created by cooling the thermistors significantly below body temperature using a burst of cryogen spray supplied when the activation button is pressed. A deficiency of this workaround is that not all of the thermistors may be cooled to the same temperature.

In current treatment devices, this lifted algorithm is used only during the initial contact of the treatment tip against the patient's skin when the cryogen spray is temporarily paused. When the treatment tip initially contacts with the skin and if the starting temperature of the treatment tip is significantly different from the skin surface temperature, the local heat flux in different regions of the contacting surfaces suddenly increases. The local heat fluxes are detected as a rapid change in the temperature reading of the nearest thermistor. When the cryogen spray is resumed, the lifted algorithm cannot be used to reliably confirm that contact is sustained at each corner of the treatment tip. Specifically, the temperature of the thermistor may not vary to a significant extent, even with high heat fluxes, because heat is removed by the evaporating cryogen concurrently with the transfer of heat from the skin to the thermistor.

Conventional treatments deliver a fixed amount of energy to the patient, as selected by the clinician, which has been calculated to provide the desired therapeutic effect by heating the tissue beneath the skin surface. However, factors such as the initial skin surface temperature profile and the electrical and thermal properties of the tissue in and around the treatment zone may influence the actual therapeutic effect imparted by the delivered energy. The temperature readings from the thermistors in conventional treatment tips are currently not used to regulate the amount of delivered energy during patient treatment because of an inability to accurately measure the skin surface temperature or to be used to estimate the subsurface dermal temperature.

What is needed, therefore, are apparatus and methods for treating skin conditions that deliver electromagnetic energy with improved thermal sensing.

### SUMMARY OF THE INVENTION

The invention is generally directed to skin condition treatment apparatus and methods that deliver electromagnetic energy with improved thermal sensing. The improved thermal sensing may eliminate or, at the least, reduce the impact associated with the artifacts of traditional temperature sensing.

In accordance with one embodiment, the treatment apparatus comprises an electrode assembly positionable adjacent to the skin surface and adapted to deliver the energy to the tissue. The assembly includes at least one thermal sensor that comprises thin or thick film traces formed on a layer of the electrode assembly and being integral therewith.

In an alternative embodiment, the thermal sensor comprises a first electrically conductive trace, a second electrically conductive trace separated from the first trace by a gap, and a body of an electrically resistive material bridging the gap. The resistive material of the body has a resistance that varies with temperature in an amount sufficient to measure the temperature.

In another aspect of the invention, a method is provided for operating a delivery device that transfers high frequency energy to tissue beneath a skin surface. The method comprises measuring a temperature difference between first and second thermal sensors in the delivery device and, based upon the measured temperature difference, determining a heat flux across a first layer separating the first and second thermal sensors. The method further comprises determining a temperature of a skin-contacting surface of a second layer separating the first thermal sensor from the skin surface based upon the determined heat flux.

In another aspect of the invention, another method is provided for operating a delivery device that transfers high frequency energy to tissue beneath a skin surface. The method comprises heating a region of the delivery device near a thermal sensor and detecting a drop in temperature with the thermal sensor when the heated region contacts the skin surface.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1 is a perspective view of a handpiece including an electrode assembly in accordance with an embodiment of the invention.

FIG. 2 is an exploded view of the electrode assembly of FIG. 1.

FIG. 3 is an end view taken generally from the perspective of line 3-3 in FIG. 2.

FIG. 4 is an enlarged view of one of the thermal sensors visible in FIG. 3.

FIG. 4A is an enlarged view similar to FIG. 4 in accordance with an alternative embodiment of the invention.

FIG. 5 is an enlarged perspective view of the thermal sensor of FIG. 4.

FIG. 6 is an enlarged top view of a thermal sensor for use with the handpiece and electrode assembly of FIG. 1 in accordance with an alterative embodiment of the invention.

FIG. 7 is an enlarged perspective view of the thermal sensor of FIG. 6.

FIG. 8 is an enlarged top view similar to FIG. 6 of a thermal sensor in accordance with an alterative embodiment of the invention.

FIG. 9 is an enlarged perspective view similar to FIG. 7 of a thermal sensor in accordance with an alterative embodiment of the invention.

FIG. 10 is an enlarged top view similar to FIG. 4 of a thermal sensor in accordance with an alterative embodiment of the invention that includes a local heating element.

FIG. 11 is a cross-sectional view of a thermal sensor in accordance with an alterative embodiment of the invention.

FIG. 12 is a schematic view of an electrical circuit in accordance with an alterative embodiment of the invention that facilitates heat flux determinations and temperature estimates of the treated target tissue.

FIG. 13A is a cross-sectional view of a thermal sensor in accordance with an alterative embodiment of the invention.

FIG. 13B is a top view of a non-patient contacting surface of an electrode including the thermal sensor of FIG. 13A.

FIG. 13C is a bottom view of a patient contacting surface of the electrode of FIG. 13A.

FIG. 14 is a top view of a flexible substrate bearing an electrode surrounded by a plurality of thermal sensors in accordance with an alterative embodiment of the invention in which the thermal sensor is shown before assembly in the treatment tip.

FIG. 14A is a cross-sectional view of a portion of the flexible substrate of FIG. 14.

FIG. 15 is a top view similar to FIG. 14 of a flexible substrate with an electrode surrounded by a plurality of thermal sensors in accordance with an alterative embodiment of the invention.

FIG. 16 is a cross-sectional view of a portion of the structure of FIG. 15 after folding and assembly in the treatment tip.

### DETAILED DESCRIPTION

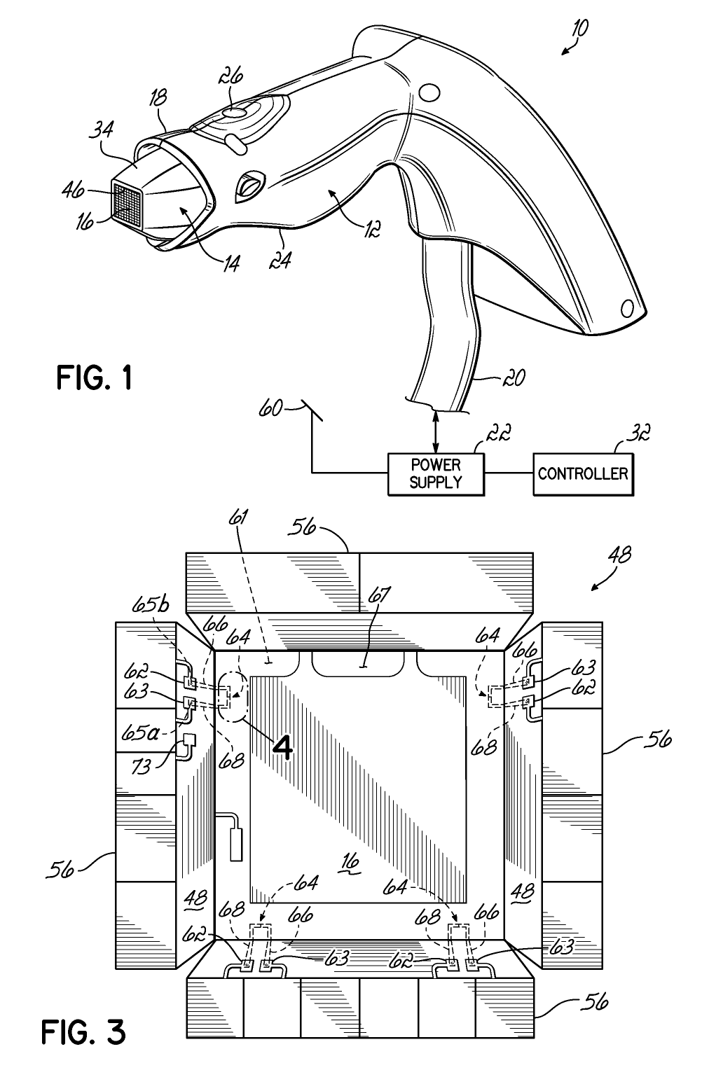

With reference to FIG. 1, a treatment apparatus or handpiece 10 includes a housing 12 typically composed of a plastic or polymer material, such as a cured polymer resin, that is molded, such as by an injection molding process, into a three-dimensional shape. Releasably coupled with the housing 12 is a delivery device in the representative form of an electrode structure or assembly 14 (i.e., treatment tip) having a leading end carrying an electrode 16, which protrudes from a shroud 18 defined at one end of the housing 12. When the electrode assembly 14 is coupled mechanically with the housing 12, the electrode 16 is exposed and visible.

Housing 12 provides a suitable interface for connection to an electrical connecting cable 20 that includes insulated and shielded conductors or wires (not shown) that electrically couple the electrode assembly 14 with a high frequency electromagnetic generator or power supply 22. Electrical connections (discussed below) inside a hollow interior of the housing 12 electrically couple the electrode assembly 14 with the high frequency power supply 22, which supplies high frequency current to the electrode 16 carried by electrode assembly 14.

Handpiece 10 includes a smoothly contoured grip portion 24 having a shape suitable for gripping and handling by the clinician. The grip portion 24 is adapted to be grasped by at least one hand of the clinician for manipulating the handpiece 10 to maneuver the electrode assembly 14 to a location proximate to a patient's skin 28 (FIG. 16). In one embodiment, a portion of the electrode 16 of electrode assembly 14 is in contact with a skin surface 29 (FIG. 16) during treatment. A target tissue 30 (FIG. 16) for the high frequency electromagnetic energy radiated from the electrode 16 lies beneath the skin surface 29. The target tissue 30 is typically the dermis of the patient's skin 28. The epidermis of the patient's skin 28 is disposed between the target tissue 30 and the skin surface 29. An activation button 26 is depressed and released for actuating a switch that controls the delivery of high frequency energy from the electrode 16 to treat the target tissue 30.

An electrical circuit (not shown) in the high frequency power supply 22 is operative to generate high frequency electrical current, typically in the radio-frequency (RF) region or band of the electromagnetic spectrum, which is transferred to the electrode 16. The operating frequency of the power supply 22 may advantageously be in the range of several hundred KHz to about 20 MHz to impart a therapeutic effect to the tissue 30. The power supply circuit in the high frequency power supply 22 converts a line voltage into drive signals having an energy content and duty cycle appropriate for the amount of power and the mode of operation that have been selected by the clinician, as understood by a person having ordinary skill in the art. High frequency energy is delivered to the patient's skin 28 and underlying tissue 30 over a short delivery cycle (e.g., about 1 second to about 10 seconds). At the conclusion of the energy delivery, the handpiece 10 is manipulated by the clinician to position the electrode assembly 14 near a different region of the patient's skin surface 29 for the performance of another treatment cycle of high frequency energy delivery.

A controller 32 is used to control the operation of the high frequency power supply 22. The controller 32 may include user input devices to, for example, adjust the applied voltage level of high frequency power supply 22 or switch between different modes of operation. The controller 32 includes a processor, which may be any suitable conventional microprocessor, microcontroller or digital signal processor, that controls and supervises the operation of the power supply 22 for regulating the power delivered from the power supply 22 to the electrode 16. Controller 32 may also include a nonvolatile memory (not shown) containing programmed instructions for the processor and may be optionally integrated into the power supply 22.

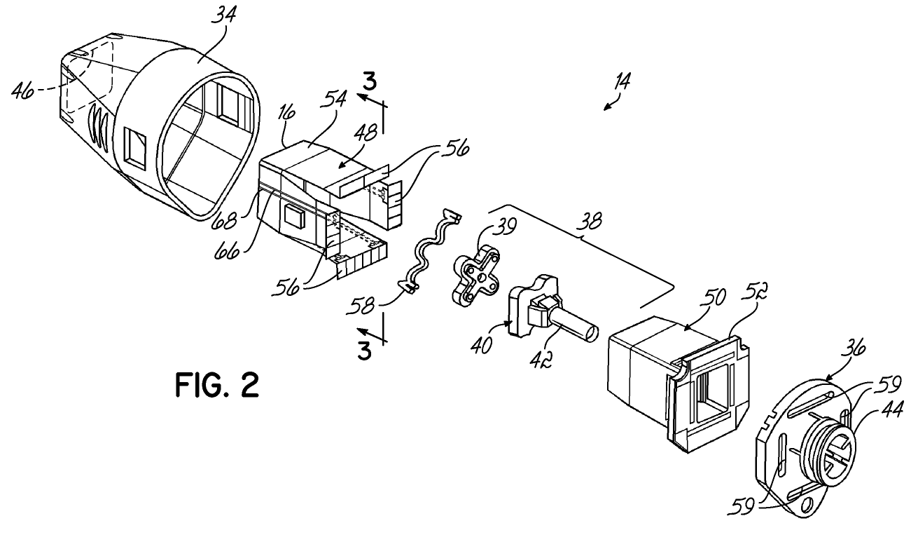

With reference to FIGS. 1 and 2, the electrode assembly 14 includes an outer shell 34 and a nipple 36 that is coupled with the open rearward end of the outer shell 34 to surround an interior cavity. A fluid delivery member 38 is configured to deliver a spray of a cryogen or similar coolant from a nozzle 39 onto the electrode 16. Extending rearwardly from a central fluid coupling member 40 is a conduit 42 having a lumen defining a fluid path that conveys a flow of the coolant to the nozzle 39. The coolant is pumped from a coolant supply (not shown) through tubing that is mechanically coupled with a fitting 44 formed on the nipple 36 and hydraulically coupled with the lumen of the conduit 42.

One purpose of the coolant spray is to pre-cool the patient's epidermis, before powering the electrode 16, by heat transfer between the electrode assembly 14 and a portion of the tissue 30, typically the patient's epidermis. As a result, the high frequency energy delivered to the tissue 30 fails to heat the epidermis to a temperature sufficient to cause significant epidermal thermal damage. Depths of tissue 30 that are not significantly cooled by pre-cooling will warm up to therapeutic temperatures resulting in the desired therapeutic effect. The amount or duration of pre-cooling may be used to select the protected depth of untreated tissue 30. The coolant spray may also be used to cool portions of the tissue 30 during and/or after heating by the transferred high frequency energy. Various duty cycles of cooling and heating by high frequency energy transfer are utilized depending on the type of treatment and the desired type of therapeutic effect. The cooling and heating duty cycles may be controlled and coordinated by the controller 32.

The electrode 16 is exposed through a window 46 defined in a forward open end of the outer shell 34. The electrode 16 may be formed as a conductive feature on a substrate 48 (FIGS. 2-4), which in the representative embodiment of the invention is a flexible sheet of dielectric material wrapped about a forward end of a support member 50. The rearward end of the support member 50 includes a flange 52 used to couple the support member 50 to the nipple 36. The flexible substrate 48 may comprise a thin base polymer (e.g., polyimide) film 54 and may include thin conductive (e.g., copper) traces or leads 56 isolated electrically from each other by small intervening gaps. Flexible substrate 48 may comprise a flex circuit having a patterned conductive (i.e., copper) foil laminated to a base polymer (or other non-conductive material) film or patterned conductive (i.e., copper) metallization layers directly deposited on a base polymer film by, for example, a vacuum deposition technique, such as sputter deposition. Flex circuits, which are commonly used for flexible and high-density electronic interconnection applications, have a construction understood by a person having ordinary skill in the art. A support arm 58 bridges the window 46 for lending mechanical support to the flexible substrate 48.

The flexible substrate 48 is wrapped or folded about the support member 50 such that the conductive leads 56 are exposed through slots 59 defined in the nipple 36. The conductive leads 56 couple the electrode 16 with the high frequency power supply 22. The conductive leads 56 may also be used to couple other structures, such as impedance or pressure sensors (not shown), with the controller 32 of high frequency power supply 22 or another control element either inside the housing 12 or external to the housing 12. A suitable treatment handpiece is shown and described in commonly-assigned U.S. application Ser. No. 11/423,068, filed Jun. 8, 2006 and published as Publication No. 20070088413 on Apr. 19, 2007, which is hereby incorporated by reference herein in its entirety.

A non-therapeutic passive or return electrode 60 (FIG. 1) is attached to a body surface of the patient that is not being treated (i.e., the patient's back) and is electrically coupled with a negative voltage polarity terminal of the high frequency power supply 22. During treatment, high frequency current flows through the bulk of the patient between the handpiece 10 and the return electrode 60 in a closed circuit. Current delivered by the handpiece 10 is returned to the high frequency power supply 22 from the return electrode 60, after having been conducted through the target tissue 30 of the patient. Because of the low current density delivered across the relatively large area of the return electrode 60, the return electrode 60 is non-therapeutic in that no significant heating is produced at its attachment site to the patient's body.

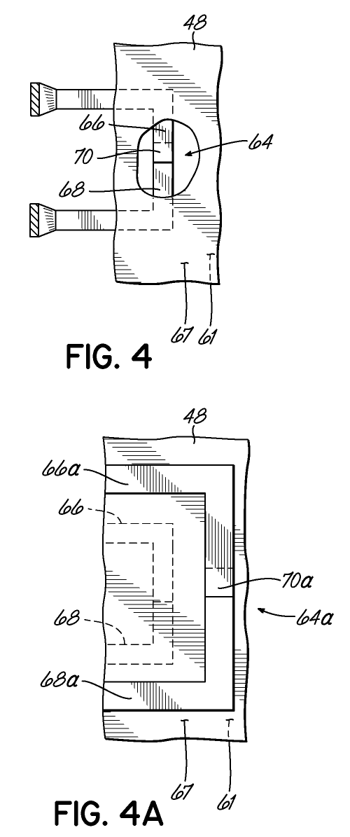

With reference to FIGS. 3 and 4 and in accordance with one embodiment of the invention, a plurality of pairs of thin or thick film trace contact pads 62, 63 are located on a non-patient contacting surface 67 of the flexible substrate 48. The trace contact pads 62, 63 are positioned within the electrode assembly 14 at locations for which the temperature is relatively constant during operation. Each of the contact pads 62, 63 is electrically coupled in continuity with a respective corresponding one of the conductive leads 56 for establishing a communications path for communicating electrical signals to the controller 32.

Electrically coupled with each of pair of contact pads 62, 63 is a respective one of a plurality of thermal sensors 64. Conductor-filled vias 65a,b (FIG. 3) extend through the flexible substrate 48 for electrically coupling each of the thermal sensors 64 with the corresponding contact pads 62, 63. Each thermal sensor 64 may directly contact the skin surface 29 or a thermal barrier (not shown) may be applied across the patient contacting surface 61 of the flexible substrate 48 to isolate the thermal sensor 64 from the skin surface 29.

The thermal sensors 64 may be configured as either thin film devices or thick film devices, as these terms are understood by a person having ordinary skill in the art. Thin film devices include at least one component of the thermal sensor 64 deposited, for example, as sputtered material onto the flexible substrate 48. Similarly, thick film devices include at least one component of the thermal sensor 64 deposited by, for example, screen printing a suitable material onto the flexible substrate 48 and curing the screen-printed material. Sputtering and screen printing techniques are understood by persons having ordinary skill in the art. The thermal sensors 64, irregardless of whether thin film or thick film devices, are significantly thinner than conventional thermistors and thermistor packages, which reduces the thermal mass and improves the time response in comparison with conventional thermistors. The thermal sensors 64 may be isolated by a thermal barrier (not shown) that significantly reduces or prevents the cryogen spray from the nozzle 39 (FIG. 2) of the fluid delivery member 38 from cooling the thermal sensors 64.

Because of the reduction in sensor thickness due to the thin film or thick film construction as compared to conventional sensor packages, the thermal sensor 64 may be positioned on a patient contacting surface 61 of the flexible substrate 48. In this instance, the thermal sensor 64 is not separated from the patient's skin surface 29 by the flexible substrate 48 and the flexible substrate 48 isolates the thermal sensors 64 against exposure to the cryogen spray. This improves detection of the actual skin temperature as the thermal sensors 64 are separated from the electrode 16 and cryogen by the thickness of the flexible substrate 48.

In an alternative embodiment of the invention, the thermal sensor 64 may be carried on a non-patient contacting surface 67 of the flexible substrate 48. An optional protective layer (not shown) may be applied across the non-patient contacting surface 67 of the flexible substrate 48 to isolate the thermal sensor 64 from cryogen. During patient treatment, the thermal sensors 64 of this alternative embodiment of the invention are separated from contact with the patient's skin surface 29 by a portion of the flexible substrate 48.

The invention contemplates that the thermal sensors 64 may be implemented by forming substrate 48 from a different dielectric, such as a ceramic or silicon, instead of a construction that consists of a flexible material of, for example, polyimide.

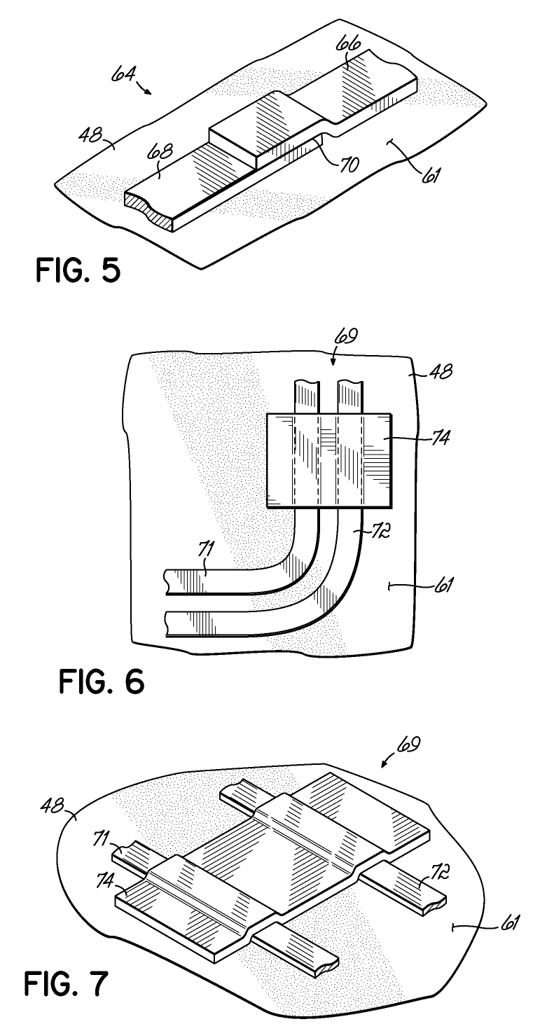

With reference to FIGS. 4 and 5 in which like reference numerals represent like features in FIGS. 1-3, each of the thermal sensors 64 may be formed on the patient contacting surface 61 in the representative form of a thermocouple including a first metal trace 66 of a first metal and a second metal trace 68 of a second dissimilar metal that overlaps or joins metal trace 66 across a relatively short overlap region or thermocouple junction 70. The metal traces 66, 68 have a good physical overlap and electrical contact across the thermocouple junction 70 to an extent that permits the thermal sensor 46 to operate as a thermocouple. The combination of the dissimilar metals of thermocouple junction 70 produces a small unique output voltage at a given temperature, which is measured and interpreted by a thermocouple thermometer in feedback circuitry (not shown) of the controller 32. The output voltage of the thermocouple junction 70 of each thermal sensor 64 is proportional to the temperature at the junction 70.

According to one embodiment, both trace contact pads 62, 63 and one of the metal traces 66, 68, for example trace 66, are made of the same material, e.g., copper. The other trace 68 is made of the second metal, e.g., constantan. This construction results in the formation of the thermocouple junction 70, as well as a reference junction at the location that via 65a intersects the trace 68. No voltage is generated at the location that via 65b intersects trace 66 because it is formed of a common metal (e.g., copper) with trace 66. A measured absolute temperature corresponding to a reference voltage measured at the reference junction 65a can be made by placing a thin or thick film thermistor 73 in a vicinity of the reference junction 65a so that feedback circuitry in the controller 32 may be used to convert the output voltage of thermocouple junction 70 to an absolute temperature measurement. The paired dissimilar metals in the metal traces 66, 68 may comprise conductors having a characteristic temperature range for temperature sensing such as, for example, the dissimilar metal pair of copper and constantan, which form a T-type thermocouple as is understood by a person having ordinary skill in the art. According to another embodiment, the reference junction 65a and the thermocouple junction 70 are located close to one another, for example, within a range of about 0.1 inch to about 4 inches of each other, or specifically within about 4 inches, within about 3 inches, within about 2 inches, or preferably within about 0.1 inch of each other.

The metal traces 66, 68 are linked by conductive leads 56 to the feedback circuitry in the controller 32. The feedback circuitry in the controller 32 receives and interprets the electrical signals communicated from the thermal sensors 64, which are indicative of the measured temperature at the location of each respective junction 70. The controller 32 uses these temperature readings to, for example, regulate the delivery of coolant to the electrode 16, to sense contact between the electrode 16 and patient's skin 28, and/or to regulate RF power delivery.

In one embodiment of the invention, metal trace 66 is composed of copper that has been etched from a conductive foil laminated with the flexible substrate 48 and metal trace 68 is composed of constantan deposited by a known technique, such as physical vapor deposition or sputtering. In this embodiment, metal trace 66 may have a thickness of about 35 μm (i.e., about 1.4 mils) and metal trace 68 may have a thickness on the order of tens of nanometers or hundreds of nanometers. Alternatively, metal trace 68 may be formed from a material other than constantan. In yet other alternative embodiments, the metal traces 66, 68 may be formed from any combination of dissimilar metals that provide a thermocouple effective to yield temperature readings across the temperature range of interest.

In another alternative embodiment of the invention, both of the metal traces 66, 68 may be formed by thick film techniques from dissimilar metals. For example, the metal traces 66, 68 may constitute screen-printed dissimilar metals, such as copper and constantan, each having a thickness of about 25 μm (i.e., about 1 mil). In yet another alternative embodiment, metal trace 66 may be composed of copper that has been etched from a conductive foil laminated with the flexible substrate 48 and metal trace 68 may be composed of constantan deposited by a known thick film technique, such as screen printing.

According to the embodiments of the invention, any combination of the traces 62, 63, 66, 68 in FIG. 4 and the traces shown in the other figures can be thin film traces, formed for example by a vacuum deposition technique such as physical vapor deposition (PVD) or sputtering. In addition and alternatively, any combination of the traces 62, 63, 66, 68 in FIG. 4 and the traces shown in the other figures can be thick film traces formed for example by a conductive layer being laminated and etched, printed, silk screened, or vacuum deposited. The thicknesses of the thin film or thick film traces produced by the various deposition techniques, as well as the deposition techniques themselves, are understood by a person having ordinary skill in the art. In another alternative embodiment of the invention, both the traces 66, 68 are thin and vacuum deposited in order to reduce the thermal mass of the active thermocouple junction.

With reference to FIG. 4A in which like reference numerals represent like features in FIGS. 1-5 and in accordance with an alternative embodiment, a thermal sensor 64a otherwise similar to thermal sensor 64 (FIGS. 3, 4) may further include another thermocouple on the non-patient contacting surface 67 of the flexible substrate 48. The additional thermocouple consists of a first metal trace 66a of a first metal and a second metal trace 68a of a second dissimilar metal that overlaps or joins metal trace 66a across a relatively short overlap region or thermocouple junction 70a. This additional thermocouple may be used for determining the local heat flux across the flexible substrate 48 in localized regions arranged about the electrode 16, as further detailed hereinbelow. The second thermocouple of thermal sensor 64, which is similar to the first thermocouple of thermal sensor 64, is also formed by a thin film or thick film technique.

With reference to FIGS. 6 and 7 in which like reference numerals represent like features in FIGS. 1-5 and in accordance with an alternative embodiment of the invention, a thin or thick film thermal sensor 69, specifically a thermistor, which may be substituted for each of the thermal sensors 64 (FIG. 3), may comprise a pair of traces 71, 72 and a body of region 74 of a material having a resistance that varies with the temperature level similar to a thermistor. Traces 71, 72 are each formed by a thin film or thick film technique from a conductive material, such as a metal like copper. Region 74 provides a resistive current path across the flexible substrate 48 between traces 71, 72 that is electrically conducting (or insulating) with a temperature dependence of resistivity (or conductivity) to an extent sufficient to measure the temperature.

Region 74 may be composed of a negative temperature coefficient material characterized by whose resistance that decreases with increasing temperature. Alternatively, region 74 may be composed of positive temperature coefficient material whose resistance increases as the temperature increases. Region 74 may be formed by either thin film or thick film techniques as understood by a person having ordinary skill in the art and, in particular, may be a thin film formed from a material having a resistance temperature coefficient (defined as the percentage change in resistance for a one degree Celsius temperature change) of a magnitude sufficient to sense measurable temperature changes over the temperatures of interest in electrode assembly 14. This configuration may be forgiving of registration errors of region 74 relative to traces 71, 72 during fabrication, which eases manufacturability. Although depicted in FIGS. 6 and 7 as formed on patient contacting surface 67, thermal sensor 69 may also be formed on non-patient contacting surface 61 of flexible substrate 48.

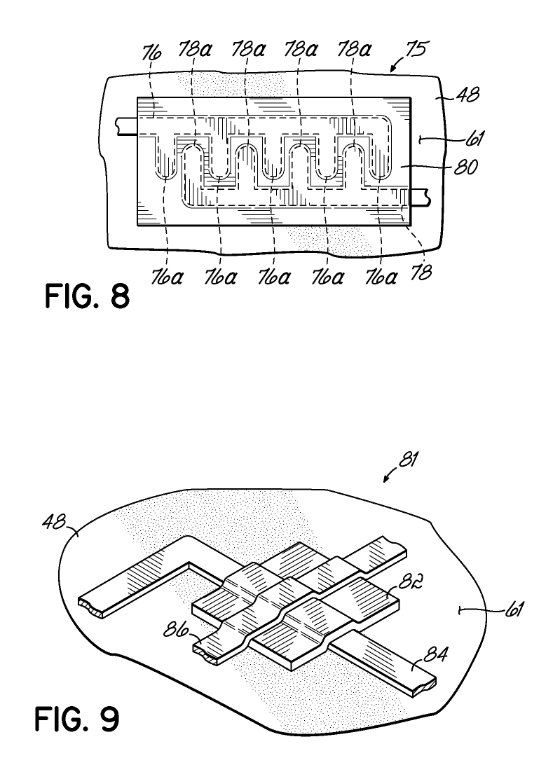

With reference to FIG. 8 in which like reference numerals represent like features in FIGS. 1-7 and in accordance with an alternative embodiment of the invention, a thermal sensor 75, which may be substituted for each of the thermal sensors 64 (FIG. 3), may comprise a pair of traces 76, 78 and a body or region 80 of a material having a resistance that varies with the temperature level, similar to the operation of a thermistor. Traces 76, 78 are each formed by a thin film or thick film technique from a conductive material, such as a metal like copper. A plurality of spaced-apart fingers 76a project from a side edge of trace 76. Similarly, trace 78 includes a plurality of fingers 78a projecting from a side edge that confronts the side edge of trace 76 from which fingers 76a project. The fingers 76a, 78a are interleaved for maximizing the active area of sensor 75 while maintaining closely-spaced traces 76, 78, which minimizes the resistance value of region 80. Region 80, which is similar to region 74 (FIGS. 6, 7), provides a resistive current path across the intervening flexible substrate 48, which is otherwise electrically insulating. Although depicted as formed on patient contacting surface 67, thermal sensor 75 may also be formed on non-patient contacting surface 61.

With reference to FIG. 9 in which like reference numerals represent like features in FIGS. 1-8 and in accordance with an alternative embodiment of the invention, a thermal sensor 81, which may be substituted for each of the thermal sensors 64 (FIG. 3), may comprise a vertical construction. To that end, a body or region 82 of a highly resistive, temperature-sensing material, which has a resistance that varies with the temperature level similar to a thermistor, vertically separates a pair of traces 84, 86 each formed from a conductive material, such as a metal like copper. The vertical configuration, which operates in a manner similar to the planar construction of FIGS. 6 and 7, conserves horizontal real estate on the flexible substrate 48 with regard to device size because of the small footprint. The resultant vertical construction is also believed to have a reduced thermal mass and to reduce thermal conduction along the traces 84, 86 in comparison with the planar construction of FIGS. 6 and 7. Although depicted as formed on patient contacting surface 67, thermal sensor 81 may also be formed on non-patient contacting surface 61. The vertical construction also provides a short path length for conduction through material region 82 to yield desired resistances from material region 82.

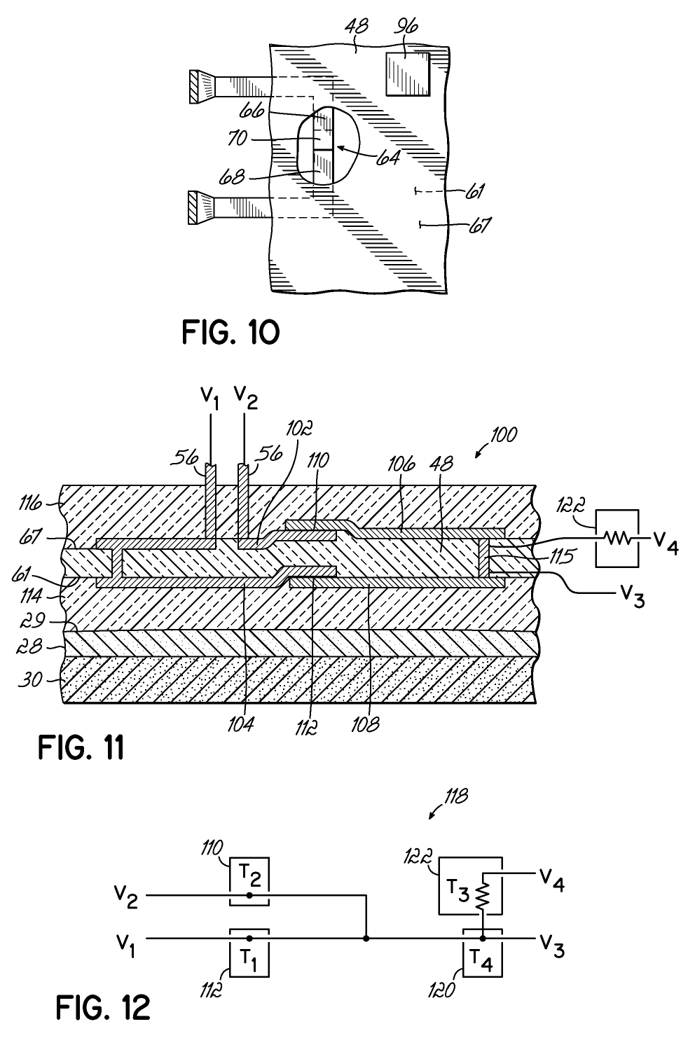

With reference to FIG. 10 in which like reference numerals represent like features in FIGS. 1-9 and in accordance with an alternative embodiment of the invention, a heating element or heater 96 may be associated in close thermal contact with each of the thermal sensors 64. Each of the heaters 96 operates by heat transfer to elevate the temperature of a respective one of the thermal sensors 64. Typically, the heater 96 must be capable of elevating the temperature of the respective thermal sensor 64 above body temperature. The invention contemplates that the heaters 96 may be used in conjunction with any of the other thermal sensors described herein. In this embodiment of the invention, each heater 96 resides on the non-patient contacting surface 61 of flexible substrate 48 and, therefore, is separated by the flexible substrate 48 from the patient's skin 28.

The heater 96 is preferably a resistive body that generates ohmic heating when an electrical current is passed through the constituent material of the heater 96. Heater 96 may be made of a patterned thin film metal, such as aluminum, copper, gold or platinum. As used herein, a “heater” may be any element or device that can be configured to actively or passively emit heat used to elevate the temperature of one of the thermal sensors 64 above body temperature.

By locally heating the thermal sensors 64 initially, as opposed to cooling thermal sensors 64, a condition in which one or more of the corners of the electrode 16 is lifted out of contact with the patient's skin 28 may be sensed without reliance upon an initial cooling below skin temperature with a burst of the cryogen spray from nozzle 39. In this embodiment of the invention, the local heating is confined by the relatively non-thermally conductive regions of the flexible substrate 48 intervening between adjacent thermal sensors 64.

The invention also contemplates that thermal sensors 69 (FIGS. 6, 7), as well as thermal sensors 75 (FIG. 8) or thermal sensors 81 (FIG. 9), may be operated in a self-heating manner such that a discrete heater, like heater 96, is not required to provide the initial heating of thermal sensor 69. This may be accomplished by momentarily applying an abnormally high voltage and current to the corresponding thermal sensor 69. The temperature reading based upon the resistance of thermal sensor 69 may be detected by the thermal sensor 69 and communicated to the controller 32 to determine when the temperature of the thermal sensor 69 has risen to the required initial temperature. Because the temperature readings from the thermal sensors 69 are independent, feedback control of the heating circuitry in controller 32 may be used to achieve uniform and repeatable starting temperatures for each thermal sensor 69.

When operated in this manner, the feedback control heating circuitry in controller 32 and thermal sensors 69 may be used to effectively determine contact between the electrode 16 and patient's skin 28. The thermal sensors 69 are held at an elevated temperature above body temperature until skin contact with skin surface 29 is established. Skin contact would be reflected by a sudden drop in the temperature of each thermal sensor 69 because of heat transfer to the patient's skin 28. Alternatively, a sudden rise in the heat demand to the thermal sensor 69 because of skin heat transfer may be used to detect skin contact.

In this alternative embodiment of the invention, the thermal sensors 69 are separated by the flexible substrate 48 from the surface 29 of the patient's skin 28, which operates as a barrier. The limited thermal mass of the thermal sensor 69 and the limited heating rate, along with the thermal insulation (i.e., low thermal conductivity) presented by the dielectric barrier of flexible substrate 48 separating the sensor 69 from the skin surface 29, operates to protect the patient's skin 28 against thermal damage.

With reference to FIG. 11 in which like reference numerals represent like features in FIGS. 1-10 and in accordance with an alternative embodiment of the invention, a thermal sensor 100, which may be substituted for each of the thermal sensors 64 (FIG. 3), comprises a pair of metal traces 102, 104 each composed of a first metal and a pair of metal traces 106, 108 each composed of a second metal dissimilar to the first metal. The dissimilar metals of traces 102 and 106 comprise a first thermocouple and overlap across a relatively short overlap region or thermocouple junction 110. Similarly, the dissimilar metals of traces 104 and 108 comprise a second thermocouple and overlap across a relatively short overlap region or thermocouple junction 112. The metal traces 102, 106 have a good electrical contact over the thermocouple junction 110, as do metal traces 104, 108 have a good electrical contact over the thermocouple junction 112. The paired dissimilar metals may comprise conductors such as, for example, copper and constantan which form a T-type thermocouple as is known to a person having ordinary skill in the art.

The thermocouple junctions 110, 112 are separated from each other by the thickness of a portion of the flexible substrate 48. Thermocouple junction 110, which is carried on the non-patient contacting surface 67, is positioned between substrate 48 and a dielectric layer 116 of a thermally-insulating and electrically-insulating material, which is optional. Dielectric layer 116 insulates the thermocouple junction 110 from the effects of cryogen spray pulses such that the temperature readings are more representative of the electrode 16. Another dielectric layer 114 of a thermally-insulating and electrically-insulating material separates thermocouple junction 112, which is carried on the patient contacting surface 61, from the skin surface 29. Dielectric layer 114 protects the thermocouple junction 112 from damage and against direct electrical contact with the skin surface 29.

The dielectric layers 114, 116 may each comprise an LPI coverlayer. Suitable LPI coverlayer materials include, but are not limited to, the Pyralux® line of photoimageable coverlayers commercially available from DuPont Electronic Materials (Research Triangle Park, N.C.) or R/Flex® line of photoimageable covercoats commercially available from Rogers Corporation (Chandler, Ariz.). The dielectric layers 114, 116 may each have a thickness of approximately 15 μm (i.e., about 0.5 mil) if constituted by LPI coverlayers.

The thermal sensor 100 is electrically coupled with one set of contact pads 62, 63. Additional thermal sensors 100 are electrically coupled with the other sets of contact pads 62, 63. A thermistor, not shown in FIG. 11, is located proximate to either of the thermocouple junctions 110, 112, to provide a reference temperature (and voltage) for such junction so that the voltage and temperature of the other junction can be determined from the voltage measurement across the leads 56. This arrangement of thermal sensors 100 permits a directional measurement of the temperature difference between adjacent junctions 110, 112. The metal traces 102, 104 are linked by conductive leads 56 to feedback circuitry in the controller 32, which receives electrical signals from the thermal sensor 100 indicative of the measured temperature at the thermocouple junctions 110, 112 and uses these temperature readings to, for example, make a heat flux measurement.

The voltage (V₁) at thermocouple junction 110 is representative of the temperature (T₁) of junction 110. The voltage (V₂) at thermocouple junction 112 is representative of the temperature (T₂) of junction 112. The voltage difference is representative of the temperature difference or thermal gradient between the thermocouple junctions 110, 112 and across the thickness of dielectric layer 48. The thermal gradient across dielectric layer 48 may be used to calculate a local heat flux based upon formulas understood by a person having ordinary skill in the art. The calculated local heat flux may be used to interpolate or extrapolate additional temperatures of interest at depths in the tissue 30 beneath the skin surface 29. By measuring the heat flux more directly, the ability to confirm contact with the skin surface 29 may be improved, even in the presence of the application of a cryogen spray.

With reference to FIG. 12 in which like reference numerals represent like features in FIGS. 1-11 and in accordance with an alternative embodiment of the invention, thermal sensor 118, which may be substituted for each of the thermal sensors 64 (FIG. 3), modifies thermal sensor 100 to further include another thermocouple junction 120. A resistive sensor 122, which could be a thermistor of the type referred to above and used in FIG. 11, is also provided. The thermocouple junction 120 and resistive sensor 122, which are each located in electrode assembly 14, cooperate with the thermocouple junctions 110, 112 to permit the calculation of heat flux through the electrode assembly 14. From the heat flux and the actual temperature measured by resistive sensor 122, an interpolated or extrapolated skin temperature can be determined.

Thermocouple junction 120 provides a reference voltage and the resistive sensor 122 measures the absolute temperature of the reference junction 120. Alternatively, a resistive sensor (not shown) capable of measuring an absolute temperature may be placed at, or near, the location of one of the thermocouple junctions 110, 112 of the thermal sensor 100. The reference junction 120 is connected with the active junction 112 to provide a first thermal temperature measurement at the active junction 112 (and first thermal sensor). As shown, this reference junction 120 is also connected to the second active junction 110 to provide a second thermal temperature measurement at the second active junction 110 (and second thermal sensor). The two active thermocouple junctions 110, 112 are located on different portions of the electrode assembly 14 so that heat flux therethrough can be determined.

The temperature of the patient contact surface 61 of substrate 48 (FIG. 3) may be calculated from the voltage at the reference junction, the measured absolute temperature at the reference junction 120, and the voltage at junction 112 using mathematical formulas familiar to a person having ordinary skill in the art, such as a one dimensional heat flux equation, based upon the electrical properties of the dissimilar thermocouple materials. With an absolute temperature measured at junction 112 and a temperature gradient measures between junctions 110 and 112, the temperature may be interpolated or extrapolated to estimate the temperature of patient's skin 28 and target tissue 30 (FIG. 16) given assumptions regarding a thickness of any coupling fluid layer and the thermal properties of the patient's skin 28 and target tissue 30. With reasonably appropriate assumptions, accurate estimates may be made of the temperatures at the skin surface 29 and at significant depths beneath the skin surface 29. Estimated subsurface temperature values may be used to determine an end point for a desired therapeutic treatment, or for feedback control of heating and cooling rates for longer duration high frequency energy treatments in which a reverse thermal gradient is established then maintained at near steady state conditions for several seconds or minutes. As a result, the amount of delivered energy may be linked to the achievement of different temperature targets in the tissue 30.

The thermal sensor 118 provides a better estimate of skin temperature than conventional thermistors found in conventional treatment tips. The temperature readings from thermal sensors 100 may be used to detect heating of a portion of the patient contact surface 61 of substrate 48 above a target temperature. If a portion of the patient contact surface 61 of substrate 48 (i.e., near an edge or a corner of electrode 16) has a non-contacting relationship with the skin surface 29, then a variation in the heat flux would be detected in one or more of the thermal sensors 100. This may be used by the controller 32 for regulating the supply of high frequency current to the electrode 16. The material or materials constituting the dielectric layers 114, 116 and flexible substrate 48 have a relatively low thermal conductivity. Although the thermal conductivity of the materials of metal traces 102, 104 and metal traces 106, 108 is significantly higher, these layers can be made extremely thin, if necessary, to limit heat transfer.

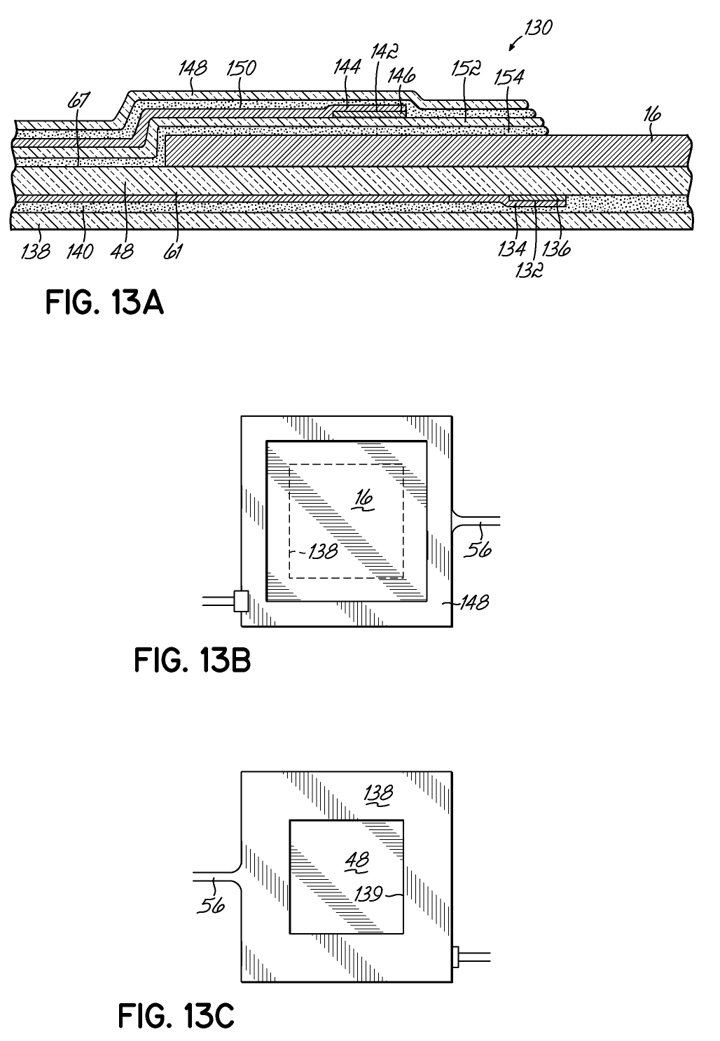

With reference to FIGS. 13A-C in which like reference numerals represent like features in FIGS. 1-12 and in accordance with an alternative embodiment of the invention, a thermal sensor 130, which may be substituted for each of the thermal sensors 100 (FIG. 11), includes a thermocouple junction 132 disposed on the inside surface of the substrate 48 that is in close thermal contact with the electrode 16. The thermocouple junction 132 is defined at the overlapping intersection of a pair of metal traces 134, 136 composed of dissimilar metals, which may be formed by physical vapor deposition by, for example, sputtering or by a thick film deposition technique, that define a thermocouple. The paired dissimilar metals of metal traces 134, 136, which are carried on the patient contacting surface 61, may comprise conductors such as, for example, copper and constantan which form a T-type thermocouple as is known to a person having ordinary skill in the art. The temperature reading measured at the thermocouple junction 132 is influenced by the temperature of the electrode 16 and the skin temperature.

A protective layer 138 of a dielectric material is bonded by an adhesive layer 140 to the substrate 48 for protecting the thermocouple junction 132 and metal traces 134, 136 against abrasion or other damage from contact and against oxidation. Layer 138 electrically isolates the thermocouple junction 132 and metal traces 134, 136 from the patient's skin surface 29 and also preferably has a thickness sufficient to reduce the capacitive high frequency pick-up from the patient to a manageable level. Protective layer 138 operates to reduce the capacitive coupling between electrode 16 to the patient's skin 28 in a region beneath an outer rim 139 of the electrode 16. Hence, the reduction in the electric field proximate to the outer rim 139 may permit a concomitant reduction in cooling. The laterally inward transfer of heat through the electrode 16 from the outer rim 139 may be improved because of the presence of protective layer 138 and adhesive layer 140 that increase the thermal resistance between the patient's skin 28 and the electrode 16 near outer rim 139.

Another thermocouple junction 142 is defined at the overlapping intersection of a pair of metal traces 144, 146 composed of dissimilar metals, which may be formed by physical vapor deposition by, for example, sputtering or by a thick film deposition technique, that form a thermocouple. The paired dissimilar metals of metal traces 144, 146, which are carried on the non-patient contacting surface 67, may comprise conductors such as, for example, copper and constantan which form a T-type thermocouple as is known to a person having ordinary skill in the art.

A protective layer 148 of a dielectric material is bonded by an adhesive layer 150 to the substrate 48 for protecting the thermocouple junction 142 and metal traces 144, 146 on one side against direct contact with the cryogen. However, the thickness of protective layer 148 is selected to permit efficient heat transfer. Similarly, another protective layer 152 of a dielectric material is bonded by an adhesive layer 154 to the substrate 48 for protecting the thermocouple junction 142 and metal traces 144, 146 on an opposite side. Protective layer 148 may be replaced by a thin layer of sputtered silicon dioxide or another dielectric material. The protective layer 152 isolates the junction 142 electrically from the conductor constituting electrode 16. Protective layer 152 operates to reduce the capacitive high frequency pickup to a manageable level. The protective layers 148, 152 may each comprise a thin LPI coverlayer. The temperature reading measured at the junction 142 is influenced by the temperature of the electrode 16 and the cryogen spray directed at the electrode 16 and thermal sensor 130.

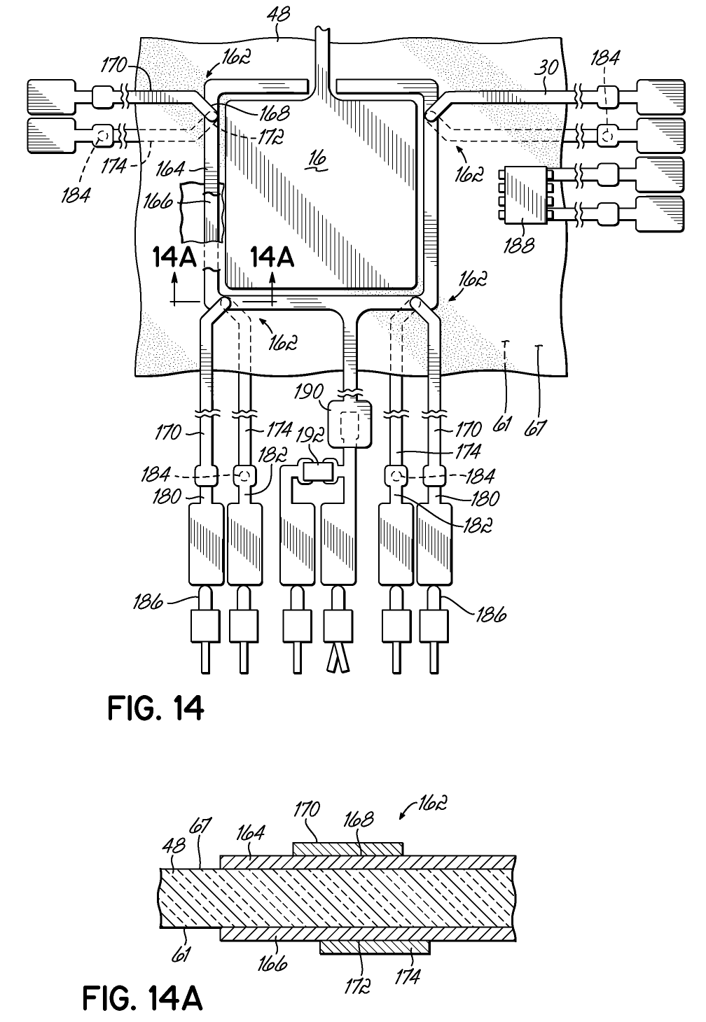

With reference to FIGS. 14 and 14A in which like reference numerals represent like features in FIGS. 1-13C and in accordance with an alternative embodiment of the invention, a plurality of substantially-identical thermal sensors 162 are arranged about a perimeter of the electrode 16 on flexible substrate 48. Almost completely encircling the perimeter of the electrode 16 on the non-patient contacting surface 67 of the flexible substrate 48 is a metal trace 164. Similarly, a metal trace 166, in a manner similar to metal trace 164, is arranged on the patient contacting surface 61 of flexible substrate 48 to almost completely encircle the perimeter of the electrode 16.

Each of the thermal sensors 162 includes a first thermocouple comprising a first thermocouple junction 168 defined at the overlapping intersection of a metal trace 170 with the metal trace 164 and a second thermocouple comprising a second thermocouple junction 172 defined at the overlapping intersection of a metal trace 174 with the metal trace 166. Thermocouple junction 168 is disposed on the non-patient contacting surface 67 of the flexible substrate 48 and thermocouple junction 172 is disposed on the patient contacting surface 61 of the flexible substrate 48. Metal traces 164, 166, 170, 174 may be formed by physical vapor deposition by, for example, sputtering or by a thick film deposition technique. Metal trace 164 and metal trace 170 are composed of dissimilar metals, as are metal traces 166 and 174. The paired dissimilar metals may comprise conductors such as, for example, copper and constantan which form a T-type thermocouple as is known to a person having ordinary skill in the art. The temperature reading measured at the junction 172 is influenced by the temperature of the electrode 16 and the skin temperature. The temperature reading measured at the junction 168 is influenced by the temperature of the electrode 16 and the cryogen spray directed at the electrode 16 and thermal sensor 162.

A conductive lead 180 on the non-patient contacting side of substrate 48 is electrically coupled with the trace 170. Similarly, another conductive lead 182 on the non-patient contacting side of substrate 48 is electrically coupled by a conductor-filled via 184 extending through substrate 48 with the metal trace 174. The conductive leads 180, 182 are each coupled with a separate electrical contact 186, such as a pogo pin. A non-volatile memory 188, such as an EEPROM, may be provided on the substrate 48 and may be used to store information relating to the electrode assembly 14. A reference voltage and a reference temperature may be supplied to the controller 32 by cooperation between a reference thermocouple 190 and a thermistor 192 that is surface mounted to the flexible substrate 48. Dielectric layers (not shown) are provided on the patient contacting and non-patient contacting sides of the flexible substrate 48.

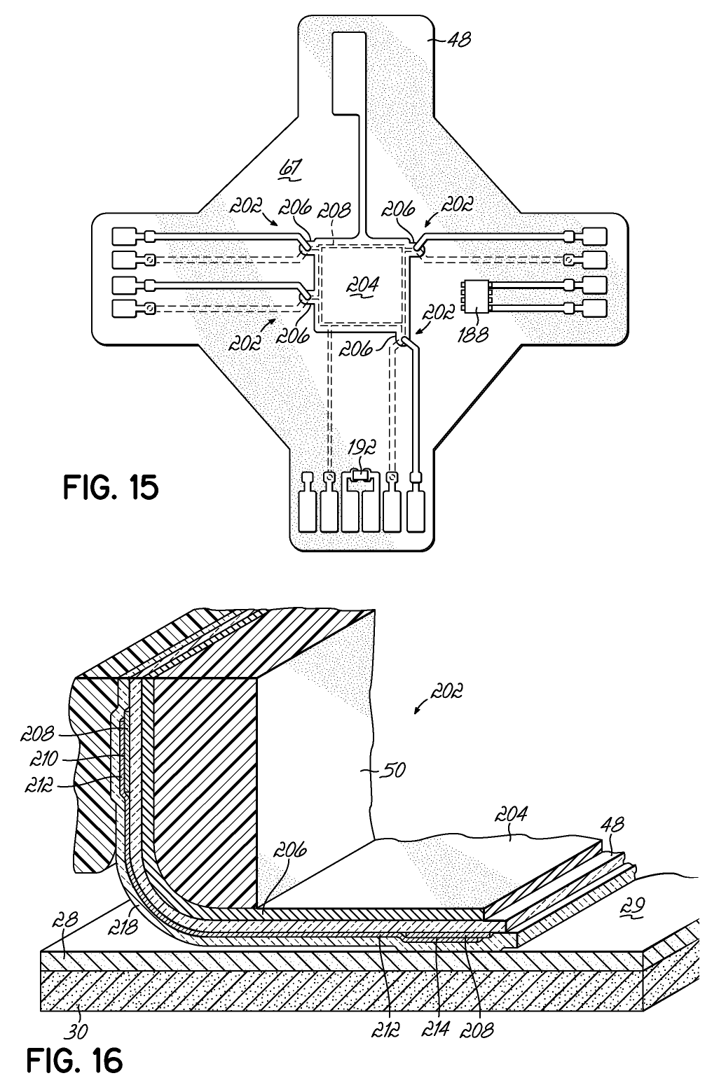

With reference to FIGS. 15 and 16 in which like reference numerals represent like features in FIGS. 1-14A and in accordance with an alternative embodiment of the invention, a plurality of substantially-identical thermal sensors 202 are arranged about a perimeter of an electrode 204, which is similar to electrode 16, disposed on flexible substrate 48. Disposed at locations about the perimeter of electrode 204 is a plurality of extensions or ears 206 that project outwardly. A metal trace 208 is separated from the electrode 204 by flexible substrate 48. Each of the thermal sensors 202 includes a first thermocouple junction 210 defined at the overlapping intersection of a metal trace 212 with a portion of metal trace 208 and a second thermocouple junction 214 defined at the overlapping intersection between metal trace 212 and another portion of metal trace 208. The thermocouple junctions 210, 214 are disposed on the patient contacting surface 61 of the flexible substrate 48. Metal traces 208 and 212 may be formed by physical vapor deposition by, for example, sputtering or by a thick film deposition technique. Metal trace 208 and metal trace 212 are composed of dissimilar metals that define a thermocouple. The paired dissimilar metals may comprise conductors such as, for example, copper and constantan which form a T-type thermocouple as is known to a person having ordinary skill in the art.

A protective layer 218 of a dielectric material is bonded to the substrate 48 for protecting the thermocouple junctions 210, 214 and metal traces 208 and 212 against abrasion or other damage from contact and against oxidation. The protective layer 218 may comprise an LPI coverlayer, as described hereinabove.

The temperature reading measured at the junction 214 may be influenced by the temperature of the electrode 16, the skin temperature, and the cryogen spray directed at the electrode 204. However, when folded about support member 50 (FIG. 2) into a configuration similar to the configuration of the flexible substrate 48 shown in FIG. 2, thermocouple junction 210 is not in a vicinity of the patient tissue and is not exposed to the cryogen spray or in the vicinity of the cryogen spray. This eliminates the impact of these influences on the temperature reading at thermocouple junction 210 and permits determinations of heat flux near the junction 214 via the difference in temperatures of the junctions 210, 214 as is understood by those skilled in the art. This embodiment may be advantageous in that heat flux through the electrode assembly can be determined from thin or thick film thermal sensors, which are formed on a common layer of the electrode assembly, as opposed to different layers as contemplated in FIGS. 11 13a, and 14a.

The temperature readings from any set of the thermal sensors 100 (FIG. 11), thermal sensors 118 (FIG. 12), thermal sensors 130 (FIGS. 13A-C), thermal sensors 162 (FIGS. 14, 14A), and thermal sensors 202 (FIGS. 15, 16) are converted into heat flux measurements. The heat flux measurements are applied to assist in one or more ways to the operation of the treatment system. Heat flux may be computed or calculated by treating each corner of the patient contact area as individual one-dimensional heat transfer problems. This simplification is reasonable because little heat is expected to conduct laterally through the thin constituent layers of these thermal sensors. The thermal conductivity of the dielectric materials is low and, although the thermal conductivity of metals is considerably higher, these layers may be thinned to limit heat transfer.

A one dimensional heat flux equation is given by:

Q=−(k·dT·A)/L

in which Q is the heat flux (measured in watts) across the dielectric layer separating the junctions, k represents the thermal conductivity (measured in Watts per meter-° K), dT is temperature difference in ° C. or ° K (i.e., T₂-T₁), A is the area involved in the thermal transfer between the treatment tip and the skin, and L is the distance that the heat must travel across the thickness of the dielectric layer. For example, a 10° C. temperature difference measured at a corner of the patient contacting surface across a dielectric layer consisting of a 25 micron thick polyimide membrane (k=0.12 W/m·K) yields a heat flux per unit area of about 48,000 W·m⁻². If this corner were not in contact with the patient's skin, the heat flux per unit area would be considerably lower.

The heat flux may be extrapolated to determine other temperatures of interest. For example, the temperature of the outer-most surface of the flex circuit construction can be calculated if the thickness and conductivity of the skin-contacting layer is known. The heat flux per unit area should be approximately the same as across the flexible substrate. The patient's skin surface temperature is approximately equal to the temperature on the thermocouple side of the coverlayer plus the temperature change across the skin-contacting layer. For example, if the skin-contacting layer is an outer LPI coverlayer having a 15 μm thickness and a conductivity of 0.10 W/m·K, a calculation using the one dimensional heat flux equation indicates that dT will be 7.2° C. The one dimensional heat flux equation may then be used to estimate the tissue temperature at any depth below the skin surface.

The invention contemplates that other mathematical equations, mathematical models, and/or simulation techniques may be used to establish the heat flux or to extrapolate the heat flux to determine other temperatures of interest, as the invention is not limited to use of the one dimensional heat flux equation. An algorithm may be implemented in the software of the treatment system controller for determining heat flux and extrapolating the heat flux.

Refinements to the calculations using the one dimensional heat flux equation may be needed to improve the accuracy of the temperature estimates. For example, the calculation may need to consider the contributions of heat removed from the thermal mass of additional components of the thermal sensor and the patient's skin may need to be considered during the rapid cooling of a pre-cool cycle. The heat input from the high frequency energy into the upper most layers of the skin may need to be considered if the temperatures are extrapolated into the skin during the treatment.

The dynamic behavior of heat flux removal may be examined as a function of skin surface temperature. Specifically, the rapid temperature changes of the pre-cool cycle might be useful to help to confirm the tissue properties used in the calculations. If the thermal mass per unit volume of the skin is known, but the thermal conductivity of the skin is unknown, it may be possible to determine the conductivity by raising the tissue to a near uniform starting temperature profile (for instance, by holding a body temperature treatment tip against the skin), then rapidly drawing heat from the skin measuring heat flux and skin surface temperature as a function of time. Other similar measurements of the dynamics of the pre-cool cycle might yield useful confirmation of tissue properties with each individual delivery of high frequency energy.

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. An apparatus for treating tissue located beneath a skin surface with electromagnetic energy, the apparatus comprising:
an electrode assembly configured to be positioned adjacent to the skin surface, the electrode assembly adapted to deliver the electromagnetic energy to the tissue, the electrode assembly including at least one thermal sensor, and the at least one thermal sensor comprising a plurality of thin film traces or thick film traces formed on a layer of the electrode assembly and being integral therewith.

2. The apparatus of claim 1 wherein the electromagnetic energy is optical energy, infrared energy, microwave energy, or radiofrequency energy.

3. The apparatus of claim 1 wherein at least one of the thin film traces or the thick film traces is defined by forming a conductor on the layer and etching away portions of the conductor.

4. The apparatus of claim 3 wherein the conductor is laminated onto the layer, printed onto the layer, silk screened onto the layer, or vacuum deposited onto the layer.

5. The apparatus of claim 1 wherein at least one of the thin film traces or the thick film traces comprises a vacuum deposited trace, the vacuum deposited trace being formed by physical vapor deposition or sputtering.

6. The apparatus of claim 1 wherein each of the thin film traces or the thick film traces is composed of a material having a thickness less than 50 microns, and the thickness of the material composing at least one of the thin film traces or the thick film traces is thinner than 10 microns.

7. The apparatus of claim 1 wherein each of the thin film traces or the thick film traces is composed of a material having a thickness less than 50 microns, and the thickness of the material composing at least one of the thin film traces or the thick film traces is thinner than 2 microns.

8. The apparatus of claim 1 wherein the thin film traces or the thick film traces define a thermistor, a thermocouple, or both.

9. The apparatus of claim 1 wherein the thin film traces or the thick film traces include a first electrically conductive trace of a first metal and a second electrically conductive trace of a second metal, the first and second traces joining across a first thermocouple junction, and the first and second metals defining a thermocouple that supplies an output voltage proportional to a temperature difference between the first thermocouple junction and a reference thermocouple junction.

10. The apparatus of claim 1 wherein the thin film traces or the thick film traces include a first trace, a second trace separated from the first trace by a gap, and a third trace composed of a material more electrically resistive than materials of the first and second traces, the third trace bridging the gap between the first and second trances, and the material of the third trace characterized by an electrical resistance that varies with temperature in an amount sufficient to measure a temperature of the third trace.

11. The apparatus of claim 10 wherein the first trace includes a first plurality of fingers and the second trace include a second plurality of fingers interleaved with the first plurality of fingers, the material in the third trace being arranged to electrically connect the first and second traces.

12. The apparatus of claim 1 wherein the electrode assembly further comprises:
a heating element positioned proximate to the thermal sensor, the heating element configured to preheat the thermal sensor before the electromagnetic energy is delivered to treat the tissue.

13. The apparatus of claim 1 wherein at least one of the thin film traces or the thick film traces is embedded and encapsulated within layers of the electrode assembly.

14. The apparatus of claim 1 wherein the electrode assembly includes a plurality of thermal sensors, at least two of the thermal sensors being formed on different layers of the electrode assembly such that first and second temperatures measured by the at least two of the thermal sensors may be used to determine a heat flux through the electrode assembly either toward or from the tissue.

15. The apparatus of claim 1 wherein the at least one thermal sensor further comprises a plurality of thermal sensors, at least two of the thermal sensors being formed on different layers of the electrode assembly such that first and second temperatures measured by the at least two of the thermal sensors may be used to determine a heat flux through the electrode assembly either toward or from the tissue.

16. The apparatus of claim 1 wherein the at least one thermal sensor further comprises first and second thermal sensors formed on different layers of the electrode assembly, the first thermal sensor located between the skin surface and the second thermal sensor, and the second thermal sensor including a thermally-conductive trace configured to provide a first temperature closer to a temperature at the skin surface than the first thermal sensor.

17. The apparatus of claim 1 wherein the at least one thermal sensor includes an active junction and a reference junction located within three inches of the active junction, and the electrode assembly includes a reference thermal sensor located adjacent to the reference junction to measure a temperature thereat so that the temperature of the active junction may be determined.

18. A method for operating a delivery device that transfers electromagnetic energy to tissue beneath a skin surface, the method comprising:
sensing a temperature difference between first and second thermal sensors in the delivery device;
determining a heat flux across the delivery device based upon the temperature difference; and
estimating a tissue temperature at a depth beneath the skin surface based upon the temperature difference and the heat flux.

19. The method of claim 18 further comprising:
measuring an absolute temperature at a location of reference thermocouple junction associated with at least one of the first and second sensors, the reference junction being located within three inches of an active junction of at least one of the first and second thermal sensors.

20. The method of claim 18 wherein the first and second thermal sensors are first and second thermocouple junctions, and sensing the temperature difference further comprises:
detecting a first voltage at the first thermocouple junction;
detecting a second voltage at the second thermocouple junction; and
comparing the first and second voltages to measure the temperature difference.

21. The method of claim 18 wherein the first and second thermal sensors are first and second bodies composed of a resistive material having a resistance that varies with temperature, and sensing the temperature difference further comprises:
detecting a first resistance of the resistive material of the first body;
detecting a second resistance of the resistive material of the second body; and
comparing the first and second resistances to measure the temperature difference.

22. A method for operating a delivery device that transfers electromagnetic energy to tissue beneath a skin surface, the method comprising:
sensing a temperature difference between first and second thermal sensors in the delivery device;
determining a heat flux across the delivery device based upon the temperature difference; and
determining a temperature of a skin-contacting surface of the delivery device based upon the heat flux.

23. The method of claim 22 further comprising:
estimating a tissue temperature at a depth beneath the skin surface based upon the temperature of the skin-contacting surface and the heat flux.

24. The method of claim 22 further comprising:
measuring an absolute temperature at a location of reference thermocouple junction associated with at least one of the first and second sensors, the reference junction being located within three inches of an active junction of at least one of the first and second thermal sensors.

25. The method of claim 22 wherein the first and second thermal sensors are first and second thermocouple junctions, and sensing the temperature difference further comprises:
detecting a first voltage at the first thermocouple junction;
detecting a second voltage at the second thermocouple junction; and
comparing the first and second voltages to measure the temperature difference.

26. The method of claim 22 wherein the first and second thermal sensors are first and second bodies composed of resistive material having a resistance that varies with temperature, and sensing the temperature difference further comprises:
detecting a first resistance of the resistive material of the first body;
detecting a second resistance of the resistive material of the second body; and
comparing the first and second resistances to measure the temperature difference.

27. A method of operating a delivery device that transfers electromagnetic energy to tissue beneath a skin surface, the method comprising:
heating a region of the delivery device near a thermal sensor; and
detecting a drop in temperature with the thermal sensor when the heated region contacts the skin surface.

28. The method of claim 27 wherein heating the region further comprises:
operating the thermal sensor to heat the region.

29. The method of claim 27 wherein heating the region further comprises:
operating a heating element adjacent to the region to heat the region.

30. The method of claim 27 further comprising:
delivering the electromagnetic energy at a radiofrequency to the tissue for heating the tissue after the temperature drop is detected.
